(19) United States
(12) Patent Application Publication
(10) Pub. No.: US 2021/0369714 A1
(43) Pub. Date: Dec. 2, 2021

(54) Prevention and Treatment of Osteoarthritis by Inhibition of Insulin Growth Factor-1 Signaling

(71) Applicant: The Regents of the University of California, Oakland, CA (US)

(72) Inventors: Daniel D. Bikle, San Francisco, CA (US); Tejal Ashwin Desai, San Francisco, CA (US); Long V. Le, San Franciso, CA (US); Yongmei Wang, San Francisco, CA (US)

(21) Appl. No.: 17/277,978

(22) PCT Filed: Sep. 26, 2019
(86) PCT No.: PCT/US2019/053285

§ 371 Date: Mar. 19, 2021

Related U.S. Application Data
(60) Provisional application No. 62/737,676, filed on Sep. 27, 2018.

(51) Int. Cl. A61K 31/519; C07K 16/22; A61K 9/00

(52) U.S. Cl. CPC A61K 31/519; A61K 9/0019; A61K 9/0092; C07K 16/22

(57) ABSTRACT

The present disclosure provides methods for treating or preventing osteoarthritis in a subject in need thereof. In certain aspects, the methods include administering an inhibitor of insulin growth factor-1 (IGF-1) signaling to a joint of the subject in an amount effective to treat or prevent osteoarthritis. In certain aspects, the osteoarthritis treated or prevented by the methods disclosed herein may to be post-traumatic osteoarthritis.

### CROSS-REFERENCE TO RELATED APPLICATIONS

This application claims the benefit of U.S. Provisional Patent Application No. 62/737,676, filed Sep. 27, 2019, which application is incorporated herein by reference in its entirety.

### GOVERNMENT SUPPORT CLAUSE

This invention was made with government support under grant No. AR055924 awarded by the National Institutes of Health. The government has certain rights in the invention.

### INTRODUCTION

43 million Americans and 15% of the world population have osteoarthritis (OA), and the incidence is increasing. 10-12% of OA is secondary to joint injury (post traumatic—PT) with an estimated cost for care in the USA of $3 billion/yr. The knee is the most frequent site of PTOA, and 50% of individuals with rupture of the anterior cruciate ligament (ACL) and/or meniscus injuries develop PTOA. Current treatments, which include surgical reconstruction of the ACL or meniscal repair, have not been able to prevent the progression to PTOA.

### SUMMARY

The present disclosure provides methods for treating or preventing osteoarthritis in a subject in need thereof. In certain aspects, the methods include administering an inhibitor of insulin growth factor-1 (IGF-1) signaling to a joint of the subject in an amount effective to treat or prevent osteoarthritis. In certain aspects, the osteoarthritis treated or prevented by the methods disclosed herein may be post-traumatic osteoarthritis.

### BRIEF DESCRIPTION OF THE DRAWINGS

FIG. 1. Schematic of loading device for ACL rupture.

FIG. 2. IGF-IR inhibitor in microrods prevents subchondral bone loss during PTOA.

FIG. 3. IGF-1R inhibitor in microrods blocks activation of IGF-1R pathways & OA development induced by ACL rupture.

FIG. 4. IGF-1R inhibitor in microrods blunts ACL rupture induced articular chondrocyte proliferation.

FIG. 5. IGF-1R in microrods decreases VEGF production induced by ACL rupture.

FIG. 6. IGF-1R inhibitor blocks MMP-13 production induced by ACL rupture.

FIG. 7. IGF1R inhibitor incorporated into microrods blocks IGF1 induced MMP production.

FIG. 8. Cumulative release of NVP-AEW541 from PEGDMA microrods.

### DETAILED DESCRIPTION

The present disclosure provides methods for treating or preventing osteoarthritis in a subject in need thereof. In certain aspects, the methods include administering an inhibitor of insulin growth factor-1 (IGF-1) signaling to a joint of the subject in an amount effective to treat or prevent osteoarthritis. In certain aspects, the osteoarthritis treated or prevented by the methods disclosed herein may be post-traumatic osteoarthritis.

Before exemplary embodiments of the present invention are described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and exemplary methods and materials may now be described. Any and all publications mentioned herein are incorporated herein by reference to disclose and describe the methods and/or materials in connection with which the publications are cited. It is understood that the present disclosure supersedes any disclosure of an incorporated publication to the extent there is a contradiction.

It must be noted that as used herein and in the appended claims, the singular forms “a”, “an”, and “the” include plural referents unless the context clearly dictates otherwise. Thus, for example, reference to “a microrod” includes a plurality of such microrods and reference to “the microrod” includes reference to one or more microrods, and so forth.

It is further noted that the claims may be drafted to exclude any element which may be optional. As such, this statement is intended to serve as antecedent basis for use of such exclusive terminology as “solely”, “only” and the like in connection with the recitation of claim elements, or the use of a “negative” limitation.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed. To the extent such publications may set out definitions of a term that conflicts with the explicit or implicit definition of the present disclosure, the definition of the present disclosure controls.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

### DEFINITIONS

The term “local administration” (or “locally administering”, “local delivery”) as used herein broadly refers to but is not limited to administration to a particular organ, tissue, or body part. Local administration includes but is not limited to intraarticular injection, intracapsule injection, surgical placement, and other local administration such as those disclosed in this invention is desirable. Local administration of a pharmaceutical composition enables delivery of a level or amount of an agent needed to treat or prevent osteoarthritis, e.g., post-traumatic osteoarthritis, without causing significant negative or adverse side effects to other tissues or organs in the body.

The term “treat”, “treating”, or “treatment” as used herein in the context of osteoarthritis, refers to reduction or resolution of one or more symptoms of osteoarthritis or prevention of further deterioration of a joint afflicted with osteoarthritis or to healing of injured or damaged tissue.

The term “prevent”, “preventing”, or “prevention” as used herein in the context of osteoarthritis, refers to reduction, delay, or absence of development of one or more symptoms of osteoarthritis.

The term “therapeutically effective amount” as used herein in the context of a pharmaceutical composition, refers to the level or amount of agent needed to treat or prevent osteoarthritis without causing significant negative or adverse side effects to the tissue where the pharmaceutical composition is administered.

The term “pharmaceutically acceptable” as used herein means biologically or pharmacologically compatible for in vivo use in animals or humans, and can mean approved by a regulatory agency of the Federal or a state government or listed in the U.S. Pharmacopeia or other generally recognized pharmacopeia for use in animals, and more particularly in humans.

Administration “administering”, as used herein, refers to contact of a therapeutically effective amount of the inhibitor(s) disclosed herein, to the subject. Administrating may be surgically or by injection or a combination thereof. Administration can be continuous or intermittent.

### METHODS

The present disclosure provides methods for treating or preventing osteoarthritis, e.g., post-traumatic osteoarthritis in a subject by administering an inhibitor of insulin growth factor-1 (IGF-1) signaling to a joint of the subject. Various steps and aspects of the methods will now be described in greater detail below.

In certain aspects, methods of the present disclosure include treating or preventing osteoarthritis in a subject in need thereof. In certain aspects, the subject may have osteoarthritis (OA) or may be at risk for developing OA. OA is also referred to as degenerative arthritis or degenerative joint disease. In certain aspects, the methods disclosed herein do not include treating or preventing arthritis other than OA, such as autoimmune conditions like rheumatoid arthritis. In certain aspects, the methods disclosed herein include treating or preventing the degeneration of joint associated with OA.

In certain aspects, OA may affect a weight-bearing joint of the subject, such as, knee, ankle, hip joint, or spine. In certain aspects, the subject may have developed OA due to aging, injury, high body mass index (BMI), obesity, gout, diabetes, joint deformity, and/or repeated motion. In certain aspects, the subject may be at risk of developing OA due to aging, injury, high body mass index (BMI), obesity, gout, diabetes, joint deformity, and/or repeated motion. In certain aspects, the injury may be a traumatic injury to a joint due to an external force, such as, motor vehicle crashes, falls, sports injuries, collisions, compressive forces, blast injury or violence. In certain aspects, the injury may be a non-traumatic injury to a joint due to illness or disease, e.g., due to an infection. In certain aspects, the OA may be post-traumatic OA (PTOA) developed due to an injury to a joint, such as, a knee joint. In certain aspects, the subject may be at risk of developing PTOA due to an injury to a joint, such as, a knee joint. In certain aspects, the injury may be an injury to the anterior cruciate ligament (ACL), such as ACL rupture or tear. In certain aspects, the injury may be an injury to the meniscus. In certain aspects, the injury may be an injury to the ACL and meniscus. In certain aspects, the treatment may be provided to a subject at the time of a surgery or after a surgery, e.g., a surgery to repair a torn ACL and/or meniscus.

In certain aspects, the methods disclosed herein include preventing development of OA in a subject. As noted in the preceding paragraph, the subject may be at risk of developing OA. In certain aspects, the subject may be at risk of developing OA following an injury to a joint. In certain aspects, the subject may be at risk of developing post-traumatic OA (PTOA) due to an injury to the joint. In certain aspect, the method for preventing development of OA in a joint of a subject due to an injury to the joint, e.g., a preventing PTOA in the subject may include administering an inhibitor of IGF-1 signaling after occurrence of injury to the joint. The administering may be performed within up to 1 year after the occurrence of injury, for example, the inhibitor of IGF-1 signaling may be administered to the joint of the subject with 1 day to 1 year after the injury, e.g., 1 day-6 months, 3 days-6months, 1 week-6 months, 2 weeks-6 months, 2 weeks-3 months, 2 weeks-1 month, e.g., within 3 days, 1 week, 3 weeks, 1 month, 2 months, 3 months, or 6 months after the occurrence of injury.

In certain aspects, preventing development of OA in a subject diagnosed as being susceptible to developing OA may be include administering an inhibitor of IGF-1 signaling to the subject after the subject is diagnosed as likely to develop OA.

In certain aspects, the methods disclosed herein include treating OA in a subject. The method may include administering an inhibitor of IGF-1 signaling to a joint of a subject diagnosed as having OA. Diagnosis of OA may be carried out using standard clinically accepted methods in the field.

In certain aspects, administering an inhibitor of IGF-1 signaling to a joint of a subject diagnosed as having OA or susceptible to developing OA may include administering the inhibitor to one or more joints. In certain aspects, a subject may have OA of one or more joints, e.g., one or more of the left knee, right knee, left shoulder, right shoulder, right hip joint, left hip joint, cervical vertebra, thoracic vertebra, lumbar vertebra, ankle, and/or a joint in the hand or foot. In such cases, the inhibitor may be administered to the one or more joints having or susceptible to developing OA. In certain aspects, the subject may be susceptible to developing PTOA due to traumatic injuring to more than one joint. In such cases, the inhibitor may be locally administered to the joints susceptible to developing PTOA.

In certain aspects, the presently disclosed methods may prevent or decrease one or more symptoms of OA. For example, the presently disclosed methods may prevent or decrease the low-grade inflammation which results in degradation of the cartilage that covers and acts as a cushion inside joints (such as knees, hips, elbows and other joints). In certain aspects, the presently disclosed methods may prevent or decrease degradation of the cartilage in joints, e.g., after an injury, such as, traumatic injury to the joint. In certain aspects, the presently disclosed methods may prevent or decrease inflammation of the joint capsule (complete envelopes surrounding the joint) often caused by breakdown products from the cartilage which are released into the joint space. In certain aspects, the presently disclosed methods may prevent or decrease new bone outgrowths, called “spurs” or osteophytes, which can form on the margins of the joints, possibly in an attempt to improve the congruence of the articular cartilage surfaces.

In certain aspects, the presently disclosed methods may prevent or decrease abnormal chondrocyte proliferation, abnormal vasculature formation, cartilage degradation, articular damage and/or osteophyte formation associated with development and/or progression of OA.

### Inhibitors of IGF-1 Signaling

According to certain embodiments, the inhibitor of IGF-1 signaling administered according to the disclosed methods may be an inhibitor of IGF-1 receptor (IGF-1R) signaling. In certain aspects, an inhibitor of IGF-1R signaling may be an antibody that binds to IGF-1 or IGF-1R and prevents IGF-1 from binding to IGF-1R. In certain embodiments, an inhibitor of IGF-1 signaling may be a small molecule that is a specific inhibitor of IGF-1R signaling. In certain aspects, the small molecule inhibitor may be an inhibitor of tyrosine kinase activity of IGF-1R. In certain aspects, the small molecule inhibitor may be a specific inhibitor of tyrosine kinase activity of IGF-1R, e.g., it may inhibit IGF-1R tyrosine kinase activity by at least 2-fold (e.g., up to 10-fold more) than it inhibits activity of another receptor, such as, insulin receptor.

In certain aspects, the inhibitor of IGF-1R signaling may be pyrrolo(2,3-d)-pyrimidine derivatives such as 7-[cis-3-(1-azetidinylmethyl)cyclobutyl]-5-[3-(phenylmethoxy)phenyl]-7H-pyrrolo[2,3-d]pyrimidin-4-amine, dihydrochloride (NVP-AEW541) or a derivative or isomer thereof. In certain aspects, the inhibitor of IGF-1R signaling may be NVP-ADW-742 (5-[3-(Phenylmethoxy)phenyl]-7-[trans-3-(1-pyrrolidinylmethyl)cyclobutyl]-7H-pyrrolo[2,3-d]pyrimidin-4-amine). In certain aspects, the inhibitor of IGF-1R signaling may be a tyrphostin, e.g., AG1024 or I-OMe-AG538. In certain aspects, the inhibitor of IGF-1 signaling may be 3-[8-Amino-1-(2-phenyl-7-quinolyl)imidazo[1,5-a]pyrazin-3-yl]-1-methyl-cyclobutanol, which is also known as linsitinib or OSI906.

In certain aspects, a small molecule IGF-1R signaling inhibitor may be a cyclolignan such as BVP 51004, picropodophyllin (PPP), BMS-554417, or XL228.

IGF1 receptor inhibitors to be used in accordance with the present methods are those described in WO 02/092599 (which is hereby incorporated by reference in its entirety) and include in particular the following compounds or salts thereof:

cis-7-(3-aminomethyl-cyclobutyl)-5-(3-benzyloxy-phenyl)-7H-pyrrolo[2,3-d]pyrimidin-4-ylamine; trans-7-(3-aminomethyl-cyclobutyl)-5-(3-benzyloxy-phenyl)-7H-pyrrolo[2,3-d]pyrimidin-4-ylamine; cis-5-(3-benzyloxy-phenyl)-7-(3-dimethylaminomethyl-cyclobutyl)-7H-pyrrolo[2,3-d]pyrimidin-4-ylamine; trans-5-(3-benzyloxy-phenyl)-7-(3-dimethylaminomethyl-cyclobutyl)-7H-pyrrolo[2,3-d]pyrimidin-4-ylamine; cis-5-(3-benzyloxy-phenyl)-7-(3-methylaminomethyl-cyclobutyl)-7H-pyrrolo[2,3-d]pyrimidin-4-ylamine; trans-5-(3-benzyloxy-phenyl)-7-(3-methylaminomethyl-cyclobutyl)-7H-pyrrolo[2,3-d]pyrimidin-4-ylamine; trans-N-{3-[4-amino-5-(3-benzyloxy-phenyl)-pyrrolo[2,3-d]pyrimidin-7-yl]-cyclobutylmethyl}-guanidine; cis-N-{3-[4-amino-5-(3-benzyloxy-phenyl)-pyrrolo [2,3-d]pyrimidin-7-yl]-cyclobutylmethyl}-gu-ιnidine; trans-N-{3-[4-amino-5-(3-benzyloxy-phenyl)-pyrrolo[2,3-d]pyrimidin-7-yl]-cyclobutylmethyl}-4-methyl-benzenesulfonamide; trans-N-{3-[4-amino-5-(3-benzyloxy-phenyl)-pyrrolo[2,3-d]pyrimidin-7-yl]-cyclobutylmethyl}-4-nitro-benzenesulfonamide; propane-2-sulfonic acid trans-{3-[4-amino-5-(3-benzyloxy-phenyl)-pyrrolo[2,3-d]pyrimidin-7-yl]-cyclobutylmethyl}-amide; ethanesulfonic acid trans-{3-[4-amino-5-(3-benzyloxy-phenyl)-pyrrolo[2,3-d]pyrimidin-7-yl]-cyclobutylmethyl}-amide; N-dimethyl-sulfamide trans-{3-[4-mino-5-(3-benzyloxy-phenyl)-pyrrolo[2,3-d]pyrimidin-7-yl]-cyclobutylmethyl}-mide; N-dimethyl-sulfamide cis-{3-[4-amino-5-(3-benzyloxy-phenyl)-pyrrolo[2,3-d]pyrimidin-7-yl]-cyclobutylmethyl}-amide; trans-{3-[4-mino-5-(3-benzyloxy-phenyl)-pyrrolo[2,3-d]pyrimidin-7-yl]-cyclobutylmethyl}-carbamic acid methyl ester; cis-{3-[4-amino-5-(3-benzyloxy-phenyl)-pyrrolo[2,3-d]pyrimidin-7-yl]-cyclobutylmethyl}-carbamic acid methyl ester; trans-{3-[4-amino-5-(3-benzyloxy-phenyl)-pyrrolo[2,3-d]pyrimidin-7-yl]-cyclobutylmethyl}-carbamic acid 2-methoxy-ethyl ester; cis-{3-[4-mino-5-(3-ben-yloxy-phenyl)-pyrrolo[2,3-d]pyrimidin-7-yl]-cyclobutylmethyl}-carbamic acid 2-methoxy-ethyl ester; trans-1-{3-[4-amino-5-(3-benzyloxy-phenyl)-pyrrolo[2,3-d]pyrimidin-7-yl]-cyclobutylmethyl}-3-ethyl-urea; cis-{3-[4-amino-5-(3-benzyloxy-phenyl)-pyrrolo[2,3-d]pyrimidin-7-yl]-cyclobutylmethyl}-3-ethyl-urea; trans-1-{3-[4-amino-5-(3-benzyloxy-phenyl)-pyrrolo[2,3- d]pyrimidin-7-yl]-cyclobutylmethyl}-3-propyl-urea; cis-1-{3-[4-amino-5-(3-benzyloxy-phenyl)-pyrrolo[2,3-d]pyrimidin-7-yl]-cyclobutylmethyl}-3-propyl-urea; trans-1-(3-[4-amino-5-(3-benzyloxy-phenyl)-pyrrolo[2,3-d]pyrimidin-7-yl]-cyclobutylmethyl}-3-isopropyl-urea; cis-1-{3-[4-amino-5-(3-benzyloxy-phenyl)-pyrrolo[2,3-d]pyrimidin-7-yl]-cyclobutylmethyl}-3-isopropyl-urea; trans-1-{3-[4-amino-5-(3-benzyloxy-phenyl)-pyrrolo[2,3-d]pyrimidin-7-yl]-cyclobutylmethyl}-3-butyl-urea; cis-1-{3-[4-amino-5-(3-benzyloxy-phenyl)-pyrrolo[2,3-d]pyrimidin-7-yl]-cyclobutylmethyl}-3-butyl-urea; trans-1-{3-[4-amino-5-(3-benzyloxy-phenyl)-pyrrolo[2,3-d]pyrimidin-7-yl]-cyclobutylmethyl}-3-tert-butyl-urea; cis-1-{3-[4-amino-5-(3-benzyloxy-phenyl)-pyrrolo[2,3-d]pyrimidin-7-yl]-cyclobutylmethyl}-3-tert-butyl-urea; trans-1-{3-[4-amino-5-(3-benzyloxy-phenyl)-pyrrolo[2,3-d]pyrimidin-7-yl]-cyclobutylmethyl}-3-benzyl-urea; trans-1-{3-[4-amino-5-(3-benzyloxy-phenyl)-pyrrolo[2,3-d]pyrimidin-7-yl]-cyclobutylmethyl}-3-(3-methyl-benzyl)-urea; cis-1-{3-[4-amino-5-(3-benzyloxy-phenyl)-pyrrolo[2,3-d]pyrimidin-7-yl]-cyclobutylmethyl}-3-(3-methyl-benzyl)-urea; cis-1-{3-[4-amino-5-(3-benzyloxy-phenyl)-pyrrolo[2,3-d]pyrimidin-7-yl]-cyclobutylmethyl}-3-(4-methoxy-benzyl)-urea; trans-1-{3-[4-amino-5-(3-benzyloxy-phenyl)-pyrrolo[2,3-d]pyrimidin-7-yl]-cyclobutylmethyl}-3-(2-mo holin-4-yl-ethyl)-urea; cis-1-{3-[4-mino-5-(3-benzyloxy-phenyl)-pyrrolo[2,3 -d]pyrimidin-7-yl]-cyclobutylmethyl}-3-(2-morpholin-4-yl-ethyl)-urea; trans-1-{3-[4-amino-5-(3-benzyloxy-phenyl)-pyrrolo[2,3 -d]pyrimidin-7-yl]-cyclobutylmethyl}-3-(2-dimethylamino-ethyl)-urea; cis-1-{3-[4-amino-5-(3-benzyloxy-phenyl)-pyrrolo[2,3-d]pyrimidin-7-yl]-cyclobutylmethyl}-3-(2-dimethylamino-ethyl)-urea; trans-1-{3-[4-amino-5-(3-benzyloxy-phenyl)-pyrrolo[2,3-d]pyrimidin-7-yl]-cyclobutylmethyl}-3-(3-moφpholin-4-yl-propyl)-urea; cis-1-{3-[4-amino-5-(3-benzyloxy-phenyl)-pyrrolo[2,3 -d]pyrimidin-7-yl]-cyclobutylmethyl}-3-(3-morpholin-4-yl-propyl)-urea; trans-1-{3-[4-amino-5-(3-benzyloxy-phenyl)-pyrrolo[2,3-d]pyrimidin-7-yl]-cyclobutylmethyl}-3-(3-dimethylamino-propyl)-urea; cis-1-{3-[4-amino-5-(3-benzyloxy-phenyl)-pyrrolo[2,3-d]pyrimidin-7-yl]-cyclobutylmethyl}-3-(3-dimethylamino-propyl)-urea; trans-{3-[4-amino-5-(3-benzyloxy-phenyl)-pyrrolo[2,3-d]pyrimidin-7-yl]-cyclobutylmethyl}-urea; cis-{3-[4-amino-5-(3-benzyloxy-phenyl)-pyrrolo[2,3-d]pyrimidin-7-yl]-cyclobutylmethyl}-urea; trans-N-{3-[4-amino-5-(3-benzyloxy-phenyl)-pyrrolo[2,3-d]pyrimidin-7-yl]-cyclobutylmethyl}-acetamide; cis-N-{3-[4-amino-5-(3-benzyloxy-phenyl)-pyrrolo[2,3-d]pyrimidin-7-yl]-cyclobutylmethyl}-acetamide; rans-N-{3-[4-amino-5-(3-benzyloxy-phenyl)-pyrrolo[2,3-d]pyrimidin-7-yl]-cyclobutylmethyl}-isobutyramide; cis-N-{3-[4-amino-5-(3-benzyloxy-phenyl)-pyrrolo[2,3-d]pyrimidin-7-yl]-cyclobutylmethyl}-isobutyramide; trans-N-{3-[4-amino-5-(3-benzyloxy-phenyl)-pyrrolo[2,3-d]pyrimidin-7-yl]-cyclobutylmethyl}-2,2-dimethyl-propionamide; cis-N-{3-[4-amino-5-(3-benzyloxy-phenyl)-pyrrolo[2,3-d]pyrimidin-7-yl]-cyclobutylmethyl}-2,2-dimethyl-propionamide; trans-N-{3-[4-amino-5-(3-benyloxy-phenyl)-pyrrolo[2,3-d]pyrimidin-7-yl]-cyclobutylmethyl}-2-morpholin-4-yl-acetamide; cis-N-{3-[4-amino-5-(3-benzyloxy-phenyl)-pyrrolo[2,3-d]pyrimidin-7-yl]-cyclobutylmethyl}-2-morpholin-4-yl-acetamide; trans-N-{3-[4-amino-5-(3-benzyloxy-phenyl)-pyrrolo[2,3-d]pyrimidin-7-yl]-cyclobutylmethyl}-2-(4-methyl-piperazin-1-yl)-acetamide; trans-5-(3-benzyloxy-phenyl)-7-(3-morpholin-4-ylmethyl-cyclobutyl)-7H-pyrrolo[2,3-d]pyrimidin-4-ylamine; trans-5-(3-benzyloxy-phenyl)-7-(3-piperidin-1-ylmethyl-cyclobutyl)-7H-pyrrolo[2,3-d]pyrimidin-4-ylamine; trans-5-(3-benzyloxy-phenyl)-7-(3-pyrrolidin-1-ylmethyl-cyclobutyl)-7H-pyrrolo[2,3-d]pyrimidin-4-ylamine (ADW); trans-1-{3-[4-amino-5-(3-benzyloxy-phenyl)-pyrrolo[2,3-d]pyrimidin-7-yl]-cyclobutylmethyl}-piperidin-4-ol; trans-7-(3-azepan-1-ylmethyl-cyclobutyl)-5-(3-benzyloxy-phenyl)-7H-pyrrolo[2,3-d]pyrimidin-4-ylamine; trans-7-(3-azetidin-1-ylmethyl-cyclobutyl)-5-(3-benzyloxy-phenyl)-7H-pyrrolo[2,3-d]pyrimidin-4-ylamine; trans-1-{3-[4-amino-5-(3-beι-zyloxy-phenyl)-pyrrolo[2,3-d]pyrimidin-7-yl]-cyclobutylmethyl}-piperidine-3-carboxylic acid amide; trans-5-(3-benzyloxy-phenyl)-7-[3-(4-pyridin-2-yl-piperazin-1-ylmethyl)-cyclobutyl]-7H-pyrrolo[2,3-d]pyrimidin-4-ylamine; trans-5-(3-benzyloxy-phenyl)-7-(3-thiomoφholin-4-ylmethyl-cyclobutyl)-7H-pyrrolo[2,3-d]pyrimidin-4-ylamine; trans-5-(3-benzyloxy-phenyl)-7-[3-(2,6-dimethyl-moφholin-4-ylmethyl)-cyclobutyl]-7H-pyrrolo[2,3-d]pyrimidin-4-ylamine; trans-(S)-1-{3-[4-amino-5-(3-benzyloxy-phenyl)-pyrrolo[2,3-d]pyrimidin-7-yl]-cyclobutylmethyl}-pyrrolidine-2-carboxylic acid amide; cis-7-(3-azepan-1-ylmethyl-cyclobutyl)-5-(3-benzyloxy-phenyl)-7H-pyrrolo[2,3-d]pyrimidin-4-ylamine; cis-1-{3-[4-amino-5-(3-benzyloxy-phenyl)-pyrrolo[2,3-d]pyrimidin-7-yl]-cyclobutylmethyl}-piperidin-4-ol; cis-4-{3-[4-amino-5-(3-benzyloxy-phenyl)-pyrrolo[2,3-d]pyrimidin-7-yl]-cyclobutylmethyl}-pipera-ine-1-carboxylic acid ethyl ester; cis-5-(3-benzyloxy-phenyl)-7-[3-(4-phenyl-piperazin-1-ylmethyl)-cyclobutyl]-7H-pyrrolo[2,3-d]pyrimidin-4-ylamine; cis-5-(3-benzyloxy-phenyl)-7-[3-(4-methyl-piperazin-1-ylmethyl)-cyclobutyl]-7H-pyrrolo[2,3-d]pyrimidin-4-ylamine; cis-5-(3-benzyloxy-phenyl)-7-(3-thiomoφholin-4-ylmethyl-cyclobutyl)-7H-pyrrolo[2,3-d]pyrimidin-4-ylamine; cis-5-(3-benzyloxy-phenyl)-7-[3-(2,6-dimethyl-moφholin-4-ylmethyl)-cyclobutyl]-7H-pyrrolo[2,3-d]pyrimidin-4-ylamine; cis-(R)-1-{3-[4-amino-5-(3-benzyloxy-phenyl)-pyrrolo[2,3-d]pyrimidin-7-yl]-cyclobutylmethyl}-pyrrolidine-2-carboxylic acid amide; cis-1-{3-[4-amino-5-(3 -benzyloxy-phenyl)-pyrrolo[2,3-d]pyrimidin-7-yl]-cyclobutylmethyl}-piperidine-3-carboxylic acid amide; trans-N-{3-[4-amino-5-(3-benzyloxy-phenyl)-pyrrolo[2,3-d]pyrimidin-7-yl]-cyclobutylmethyl}-2-ethoxy-acetamide; trans-N-{3-[4-amino-5-(3-benzyloxy-phenyl)-pyrrolo[2,3-d]pyrimidin-7-yl]-cyclobutylmethyl}-2-(2-methoxy-ethoxy)-acetamide; trans-1-{3-[4-mino-5-(3-benzyloxy-phenyl)-pyrrolo[2,3-d]pyrimidin-7-yl]-cyclobutylmethyl}-3-methyl-urea; cis-1-{3-[4-amino-5-(3-benzyloxy-phenyl)-pyrrolo[2,3-d]pyrimidin-7-yl]-cyclobutylmethyl}-3-methyl-urea; trans-pyrrolidine-1-carboxylic acid {3-[4-amino-5-(3-benzyloxy-phenyl)-pyrrolo[2,3-d]pyrimidin-7-yl]-cyclobutylmethyl}-amide; trans-piperidine-1-carboxylic acid {3-[4-amino-5-(3-benzyloxy-phenyl)-pyrrolo[2,3-d]pyrimidin-7-yl]-cyclobutylmethyl}-amide; trans-moφholine-4-carboxylic acid {3-[4-amino-5-(3-benzyloxy-phenyl)-pyrrolo[2,3-d]pyrimidin-7-yl]-cyclobutylmethyl}-amide; trans-3-{3-[4-amino-5-(3-benzyloxy-phenyl)-pyrrolo[2,3-d]pyrimidin-7-yl]-cyclobutylmethyl}-1,1-dimethyl-urea; trans-4-methyl-piperazine-1-carboxylic acid {3-[4-amino-5-(3-benzyloxy-phenyl)-pyrrolo[2,3-d]pyrimidin-7-yl]-cyclobutylmethyl}-amide; trans-3-{3-[4-mino-5-(3-benzyloxy-phenyl)-pyrrolo[2,3-d]pyrimidin-7-yl]-cyclobutylmethyl}-1,1-diethyl-urea; trans-{3-[4-amino-5-(3-benzyloxy-phenyl)-py-τolo[2,3-d]pyrimidin-7-yl]-cyclobutylmethyl}-carbamic acid 2-diethylamino-ethyl ester; trans-{3-[4-amino-5-(3-benzyloxy-phenyl)-pyrrolo[2,3-d]pyrimidin-7-yl]-cyclobutylmethyl}-carbamic acid 2-moφholin-4-yl-ethyl ester; trans-{3-[4-amino-5-(3-benzyloxy-phenyl)-pyrrolo[2,3-d]pyrimidin-7-yl]-cyclobutylmethyl}-carbamic acid 2-(4-methyl-piperazin-1-yl)-ethyl ester; trans-{3-[4-amino-5-(3-ber-zyloxy-phenyl)-pyrrolo[2,3-d]pyrimidin-7-yl]-cyclobutylmethyl}-carbamic acid 2-dimethylamino-ethyl ester; trans-{3-[4-amino-5-(3-benzyloxy-phenyl)-pyrrolo[2,3-d]pyrimidin-7-yl]-cyclobutylmethyl}-carbamic acid ethyl ester; trans-4-{3-[4-amino-5-(3-benzyloxy-phenyl)-pyrrolo[2,3-d]pyrimidin-7-yl]-cyclobutylmethyl}-piperazine-1-carboxylic acid ethyl ester; cis-5-(3-benzyloxy-phenyl)-7-(3-pyrrolidin-1-ylmethyl-cyclobutyl)-7H-pyrrolo[2,3-d]pyrimidin-4-ylamine; cis-7-(3-azetidin-1-ylmethyl-cyclobutyl)-5-(3-benzyloxy-phenyl)-7H-pyrrolo[2,3-d]pyrimidin-4-ylamine; trans-3-[4-Amino-5-(3-benzyloxy-phenyl)-6-bromo-pyrrolo[2,3-d]pyrimidin-7-yl]-cyclobutanecarboxylic acid methylester; trans-3-[4-Amino-5-(3-benzyloxy-phenyl)-6-methyl-pyrrolo[2,3-d]pyrimidin-7-yl]-cyclobutanecarboxylic acid methyl ester; trans-{3-[4-Amino-5-(3-ben-yloxy-phenyl)-6-methyl-pyrrolo[2,3-d]pyrimidin-7-yl]-cyclobutyl}-methanol; cis-3-[4-Amino-5-(3-benzyloxy-phenyl)-6-bromo-pyrrolo[2,3-d]pyrimidin-7-yl]-cyclobutanecarboxylic acid methyl ester; cis-3-[4-Amino-5-(3-benzyloxy-phenyl)-6-methyl-pyrrolo[2,3-d]pyrimidin-7-yl]-cyclobutanecarboxylic acid methyl ester; cis-{3-[4-Amino-5-(3-benzyloxy-phenyl)-6-methyl-pyrrolo[2,3-d]pyrimidin-7-yl]-cyclobutyl}-methanol; cis-3-[4-Amino-5-(3-benzyloxy-phenyl)-6-ethyl-pyrrolo[2,3-d]pyrimidin-7-yl]-cyclobutanecarboxylic acid methyl ester; trans-3-[4-Amino-5-(3-benzyloxy-phenyl)-6-ethyl-pyrrolo[2,3-d]pyrimidin-7-yl]-cyclobutanecarboxylic acid methyl ester; cis-{3-[4-Amino-5-(3-benzyloxy-phenyl)-6-ethyl-pyrrolo[2,3-d]pyrimidin-7-yl]-cyclobutyl}-methanol; trans-{3-[4-Amino-5-(3-benzyloxy-phenyl)-6-ethyl-pyrrolo[2,3-d]pyrimidin-7-yl]-cyclobutyl}-methanol; trans-5-(3-Benzyloxy-phenyl)-6-methyl-7-(3-pyrrolidin-1-ylmethyl-cyclobutyl)-7H-pyrrolo[2,3-d]pyrimidin-4-ylamine; trans-5-(3-Benzyloxy-phenyl)-6-methyl-7-[3-(4-methyl-piperazin-1-ylmethyl)-cyclobutyl]-7H-pyrrolo[2,3-d]pyrimidin-4-ylamine; trans-1-{3-[4-Amino-5-(3-benzyloxy-phenyl)-6-methyl-pyrrolo[2,3-d]pyrimidin-7-yl]-cyclobutylmethyl}-piperidin-4-ol; trans-7-(3-Azetidin-1-ylmethyl-cyclobutyl)-5-(3-benzyloxy-phenyl)-6-methyl-7H-pyrrolo[2,3-d]pyrimidin-4-ylamine; trans-5-(3-Benzyloxy-phenyl)-6-methyl-7-{3-[(tetrahydro-pyran-4-ylamino)-methyl]-cyclobutyl}-7H-pyrrolo[2,3-d]pyrimidin-4-ylamine; trans-((R)-1-{3-[4-Amino-5-(3-benzyloxy-phenyl)-6-methyl-pyrrolo[2,3-d]pyrimidin-7-yl]-cyclobutylmethyl}-pyrrolidin-2-yl)-methanol; cis-5-(3-Benzyloxy-phenyl)-6-methyl-7-(3-pyrrolidin-1-ylmethyl-cyclobutyl)-7H-pyrrolo[2,3-d]pyrimidin-4-ylamine; cis-7-(3-Azetidin-1-ylmethyl-cyclobutyl)-5-(3-benzyloxy-phenyl)-6-methyl-7H-pyrrolo[2,3-d]pyrimidin-4-ylamine; cis-1-{3-[4-Amino-5-(3-benzyloxy-phenyl)-6-methyl-pyrrolo[2,3-d]pyrimidin-7-yl]-cyclobutylmethyl}-piperidin-4-ol; cis-((R)-1-{3-[4-Amino-5-(3-benzyloxy-phenyl)-6-methyl-pyrrolo[2,3-d]pyrimidin-7-yl]-cyclobutylmethyl}-pyrrolidin-2-yl)-methanol; cis-5-(3-Benzyloxy-phenyl)-6-ethyl-7-(3-pyrrolidin-1-ylmethyl-cyclobutyl)-7H-pyrrolo[2,3-d]pyrimidin-4-ylamine; cis-5-(3-Benzyloxy-phenyl)-6-ethyl-7-[3-(4-methyl-piperazin-1-ylmethyl)-cyclobutyl]-7H-pyrrolo[2,3-d]pyrimidin-4-ylamine; cis-1-{3-[4-Amino-5-(3-benzyloxy-phenyl)-6-ethyl-pyrrolo[2,3-d]pyrimidin-7-yl]-cyclobutylmethyl}-piperidin-4-ol; cis-((R)-1-{3-[4-Amino-5-(3-benzyloxy-phenyl)-6-ethyl-pyrrolo[2,3-d]pyrimidin-7-yl]-cyclobutylmethyl}-pyrrolidin-2-yl)-methanol; and cis-5-(3-Benzyloxy-phenyl)-6-ethyl-7-{3-[(tetrahydro-pyran-4-ylamino)-methyl]-cyclobutyl}-7H-pyrrolo[2,3-d]pyrimidin-4-ylamine. In certain aspects, the inhibitor may be trans-5-(3-benzyloxy-phenyl)-7-(3-pyrrolidin-1-ylmethyl-cyclobutyl)-7H-pyrrolo[2,3-d]pyrimidin-4-ylamine (ADW-742). The preparation of these compounds is described in WO 02/092599. In some cases, the inhibitor is imatinib, dasatinib, tofacitinib, as well as other tyrosine kinase inhibitors (TKIs), including salts or esters thereof.

Reduction in IGF-1 signaling may be achieved by reducing IGF-1 levels in a subject by administration of small molecules, and inhibition of IGF-1 transcription and/or translation. For example, IGF-1 levels are reduced by somatostatin and analogs thereof, by inhibitors of growth hormone production, by agents which decrease growth hormone bioavailability, by inhibitors of the growth hormone receptor and/or by inhibitors of signaling cascades downstream of the growth hormone receptor. Somatostatin analogs include octreotide (SMS 201-995), lanreotide, depreotide, vapreotide (RC-160), somatuline (BIM 23014), TT-232, AN-238. Other suitable somatostatin analogs are those disclosed in U.S. Pat. No. 6,465,613 the contents of which are hereby incorporated by reference. Inhibitors of growth hormone production include antisense oligodeoxynucleotides (ODNs) or small inhibitory RNA (siRNA) against the mRNA transcript for the growth hormone molecule. Agents which decrease growth hormone bioavailability include neutralizing antibodies against growth hormone, soluble growth hormone receptors or other proteins which can be engineered to bind growth hormone with higher affinity than its receptor in target tissues. Inhibitors of the growth hormone receptor include neutralizing antibodies, inhibitory peptides or small molecule inhibitors which prevent growth hormone from binding to its receptor and/or activating its downstream signaling pathways in target tissues. Inhibitors of growth hormone receptor downstream signaling cascade include antisense ODNs, siRNA constructs, peptides, small molecule inhibitors or other strategies that can block the signaling pathways which are stimulated by growth hormone receptor and which can lead to increased production of IGF-1.

IGF-1 transcription is inhibited, for example, by targeting nucleotide sequences complementary to the regulatory region of the IGF nucleic acid (e.g., the IGF-1 promoter and/or enhancers) to form triple helical structures that prevent transcription of the IGF-1 gene in target cells. Alternatively, IGF translation is inhibited by an antisense nucleic acid, such as a ribozyme. Ribozymes are catalytic RNA molecules with ribonuclease activity that are capable of cleaving a single-stranded nucleic acid, such as an mRNA, to which they have a complementary region. A ribozyme having specificity for an IGF-1-encoding nucleic acid can be designed based upon the nucleotide sequence of an IGF-1 cDNA.

In certain aspects, the inhibitor of IGF-1R signaling may be a monoclonal antibody that binds to IGF-1R ligand such as IGF-1 or IGF-2 and prevents them from binding to IGF-1R. In certain aspects, the monoclonal antibody may be MEDI-573.

In certain aspects, the inhibitor of IGF-1R signaling may be a monoclonal antibody that binds to IGF-1R prevents it from binding to its ligand and/or induces receptor degradation and/or causes antibody dependent cell-mediated cytotoxicity (ADCC, if IgG1). In certain aspects, the monoclonal antibody may be cixutumumab (IMC-A12), figitumumab (CP-751,871), Dalotuzumab (MK-0646; h7C10), Ganitumab (AMG 479), R1507, SCH 717454 (19D12), AVE1642 (EM164), or BIIB022.

### Compositions of Inhibitors of IGF-1 Signaling

The inhibitors of IGF-1 signaling disclosed herein may be present in pharmaceutical compositions for local administration. The pharmaceutical composition may additionally include a pharmaceutically acceptable diluents, carriers or excipients. In certain embodiments, a pharmaceutical composition also includes at least one additional prophylactic or therapeutic agent. Suitable pharmaceutically acceptable or physiologically acceptable diluents, carriers or excipients include, but are not limited to, antioxidants (e.g., ascorbic acid and sodium bisulfate), preservatives (e.g., benzyl alcohol, methyl parabens, ethyl or n-propyl, p-hydroxybenzoate), emulsifying agents, suspending agents, dispersing agents, solvents, fillers, bulking agents, detergents, buffers, vehicles, and/or diluents. For example, a suitable vehicle may be physiological saline solution or citrate buffered saline, possibly supplemented with other materials common in pharmaceutical compositions for parenteral administration. Neutral buffered saline or saline mixed with serum albumin are further exemplary vehicles.

In certain aspects, inhibitors of IGF-1 signaling may be formulated for sustained release from biocompatible, biodegradable, polymeric nanoparticles and/or biocompatible, biodegradable, polymeric particle formulations, such as, nanorods or microrods. The term “particles” as used herein broadly refers to nanoparticles (e.g., nanorods), microparticles (e.g., microrods), or other sized particles. The particles and polymer particles described herein can comprise nanoparticles, microparticles, larger particles, or combinations of particle sizes.

In some aspects, the IGF-1 signaling inhibitors may be provided in PLGA nanoparticle and/or microparticle formulations. PLGA nanoparticle and/or microparticle formulations provided herein are suitable for local administration via injection (such as intraarticular).

Any pharmaceutically acceptable biodegradable polymer known in the art can be used to provide IGF-1 signaling inhibitor containing particles as described herein. Suitable biodegradable polymers include but are not limited polylactic acid (PLA), polyglycolic acid (PGA), poly(ε-caprolactone) (PCL), poly(ethylene glycol) diacrylate (PEGDA), poly(ethylene glycol) dimethacrylate (PEGDMA), SU-8, poly-α-hydroxy acid esters such as polylactic acid (PLLA or DLPLA), polyglycolic acid, polylactic-co-glycolic acid (PLGA), polylactic acid-co-caprolactone; poly (ester-co-amide) copolymers; poly (block-ethylene oxide-block-lactide-co-glycolide) polymers (PEO-block-PLGA and PEO-block-PLGA-block-PEO); polyethylene glycol and polyethylene oxide, poly (block-ethylene oxide-block-propylene oxide-block-ethylene oxide), polyanhydrides, polyphosphazenes, polyaminoacids, etc. In particular embodiments, the biodegradable polymer is PLGA with molar compositions having a lactic acid (LA): glycolic acid (GA) ratio ranging from 100:0 to 50:50 molecular weight of 7 kDa-100 kDa. Additionally, two or more forms of the biocompatible, biodegradable PLGA can be employed, one being the more hydrophobic end-capped polymer with the terminal residues functionalized as esters, and the other being the more hydrophilic uncapped polymer with the terminal residues existing as carboxylic acids.

It is appreciated by one skilled in the art that the degradation rates of said PLGA particles and drug release from said particles can be influenced by different parameters: (i) the molecular weight: increasing the molecular weight of conventional PLGAs from 7 to 100 kDa, degradation rates were reported to range from several weeks to several months; (ii) the ratio of lactic acid (LA) to glycolic acid (GA): PLGA with a higher content of LA are less hydrophilic, absorb less water and subsequently degrade more slowly, as a consequence of the presence of methyl side groups in PLA making it more hydrophobic than PGA. An exception to this rule is the copolymer 50:50 which exhibits the faster degradation; (iii) stereochemistry: mixtures of D and L lactic acid monomers are most commonly used for PLGA fabrication, as the rate of water penetration is higher in amorphous D,L regions, leading to accelerated PLGA degradation; and (iv) end-group functionalization: polymers that are end-capped with esters (as opposed to the free carboxylic acid) demonstrate longer degradation half-lives. Moreover, the shape of the PLGA particle (e.g., particle size) strongly affects PLGA degradation behavior depending on the accessibility of water. In addition, acidic surrounding media accelerate PLGA degradation due to autocatalysis.

These inhibitor containing PLGA nanoparticles and/or microparticles and formulations thereof are collectively referred to herein as “inhibitor/PLGA particles” and “inhibitor/PLGA particle formulations,” where these terms are used interchangeably. “inhibitor/polymer particles” include “inhibitor/PLGA particles” as well as inhibitor particles formulated with other polymers. The target for the general composition of the inhibitor/PLGA particles described herein will generally range from 10 to 90% inhibitor in the composition, % of polylactic acid in the polylactic acid polyglycolic acid (PLGA) copolymer can be 0-100%, e.g., about 30% inhibitor, in 50/50 PLGA with molecular weight of 7-17 kDa, inherent viscosity 0.16-0.24 dL/g, and the average particle size of the nanoparticles is 20 nm-100 μm.

The compositions and formulation can optionally contains a viscosity enhancer such as hyaluronic acid.

The terms “biodegradable” and “biodegradable polymer” refer to biodegradable technology utilized by the bio-medical community. Biodegradable polymers are classified into three groups: medical, ecological, and dual application, while in terms of origin they are divided into two groups: natural and synthetic. The polymer (meaning a material composed of molecules with repeating structural units that form a long chain) is used to encapsulate or form a reservoir for a drug prior to injection in or administration to the body and is frequently based on lactic acid, a compound normally produced in the body, and is thus able to be excreted naturally. The coating is designed for controlled release over a period of time, reducing the number of injections or administrations required and maximizing the therapeutic benefit. Once introduced into the body, biodegradable polymers require no retrieval or further manipulation and are degraded into soluble, non-toxic by-products. Different polymers degrade at different rates within the body and therefore polymer selection can be tailored to achieve desired release rates. The term “biodegradable polymer” also refers to a polymer or polymers which degrade in vivo, and wherein erosion of the polymer or polymers over time occurs concurrent with or subsequent to release of the therapeutic agent. The terms “biodegradable” and “bioerodible” are equivalent and are used interchangeably herein. A biodegradable polymer may be a homopolymer, a copolymer, or a polymer comprising more than two different polymeric units.

The concentration of the IGF-1 signaling inhibitor or the IGF-1 signaling inhibitor content in the formulations of the present disclosure will depend on the selected route of administration and dosage form, but will generally range from about 10 to about 90% (w/w). The average range of IGF-1 signaling inhibitor content of the present disclosure is preferably from about 10% to 90% by weight of the pharmaceutical formulation (w/w). In some embodiments, the inhibitor/polymer particles are about 10%-25%(w/w), about 10%-35% (w/w), about 10%-50% (w/w), about 15%-25% (w/w), about 15%-40% (w/w), about 15%-65% (w/w), about 20%-65%(w/w), about 20%-90% (w/w), about 25%-85% (w/w), about 30%-90% (w/w), about 40%-60% (w/w), about 40%-75% (w/w), about 40%-90% (w/w), about 50%-75% (w/w), about 50%-90% (w/w), about 60%-85% (w/w) and about 60%-90% (w/w). Examples of useful polymeric materials include, without limitation, such materials derived from and/or including organic esters and organic ethers, which when degraded result in physiologically acceptable degradation products.

In some embodiments, copolymers of glycolic acid (GA) and lactic acid (LA) are used, where the rate of biodegradation is controlled by the ratio of glycolic acid to lactic acid. The most rapidly degraded copolymer has roughly equal amounts of glycolic acid and lactic acid. Homopolymers, or copolymers having ratios other than equal, are more resistant to degradation. The ratio of glycolic acid to lactic acid will also affect the brittleness of the pharmaceutical composition, where a more flexible composition is desirable for larger geometries. PLGA with a higher content of LA are less hydrophilic, absorb less water and subsequently degrade more slowly, as a consequence of the presence of methyl side groups in PLA making it more hydrophobic than PGA. An exception to this rule is the copolymer 50:50 which exhibits the faster degradation. Broadly the % of poly lactic acid (LA) in the PLGA copolymer is 50-100%, preferably about 15-85%, more preferably about 35-75%. The ratio of lactic acid (LA) to glycolic acid (GA) in the polylactic acid polyglycolic acid (PLGA) copolymer can be 0-100%. In some embodiments, the ratio of LA:GA is about 85:15, the ratio of LA:GA is about 75:25, the ratio of LA:GA is about 65:35, the ratio of LA:GA is about 60:40, the ratio of LA:GA is about 55:45, the ratio of LA:GA is 50:50, the ratio of LA:GA is 45:65, the ratio of LA:GA is 40:60, the ratio of LA:GA is about 35:65, the ratio of LA:GA is about 30:70, the ratio of LA:GA is about 25:75. In a particular embodiment, an approximately 75:25 PLGA copolymer is used. In a particular embodiment, an approximately 50:50 PLGA copolymer is used.

The biodegradable polymer matrix of the present invention may comprise a mixture of two or more biodegradable polymers. For example, the pharmaceutical composition may comprise a mixture of a first biodegradable polymer and a different second biodegradable polymer. One or more of the biodegradable polymers may have terminal acid groups. Release of a drug from an erodible polymer is the consequence of several mechanisms or combinations of mechanisms. Some of these mechanisms include desorption from the implants surface, dissolution, diffusion through porous channels of the hydrated polymer and erosion. Erosion can be bulk or surface or a combination of both. As discussed herein, the matrix of the pharmaceutical composition may release drug at a rate effective to sustain release of an amount of the inhibitor for more than one week after administration into desired location. In certain embodiments, therapeutic amounts of the inhibitor are released for more than about one month, and even for about six months or more.

Another example of the long acting, biodegradable pharmaceutical composition comprises a IGF-1 signaling inhibitor with a biodegradable polymer matrix that comprises a single type of polymer. For example, the biodegradable polymer matrix may consist essentially of a polycaprolactone. The polycaprolactone may have a molecular weight between about 10 and about 20 kilodaltons, such as about 15 kilodaltons. These formulations are capable of providing a nearly linear release rate for at least about 70 days, or for at least about 50 days, or for at least about 30 days, for at least about 15 days, or for at least about 7 days. In some embodiments, the inhibitor/PLGA particles or inhibitor particles have a mean diameter in the range of about 0.02 to 100μm, for example, as detected by laser light scattering methods. In some embodiments, the particles have a mean diameter in the range of about 20-100 nm, about 20-200 nm, about 40-400 nm, about 40-600 nm, about 60-800 nm, about 60-1000 nm, about 200 nm-2 μm, about 400 nm-2 μm, about 600 nm-4 μm, about 600 nm-6 μm, about 800 nm-4 μm, about 800 nm-6 μm, about 800 nm-1 μm, about 1 μm-20 μm, about 1 μm-40 μm, about 10 μm-30 μm, about 20 μm-40 μm, about 20 μm-60 μm, about 30 μm-60 μm, about 30 μm-80 μm, about 40 μm-60 μm, about 50 μm-80 μm, about 40 μm-80 μm, about 40 μm-90 μm, about 40 μm-100 μm. It is understood that these ranges refer to the mean diameter of all particles in a given population. The diameter of any given individual particle could be within a standard deviation above or below the mean diameter.

In some embodiments, the inhibitor/PLGA particles or inhibitor particles are administered in a formulation having a viscosity in the range of about 2.0 centipoise (cP) to about 4 cP. In some embodiments the formulation has a viscosity in the range of about 2.7 cP to about 3.5 cP. In some embodiments, the particles are administered in a formulation having a viscosity in the range of about 2.8 cP to about 3.5 cP, about 2.9 cP to about 3.5 cP, about 3.0 cP to about 3.5 cP, about 3.1 cP to about 3.5 cP, about 3.2 cP to about 3.5 cP, about 3.3 cP to about 3.5 cP, about 3.4 cP to about 3.5 cP, about 2.8 cP to about 3.2 cP, about 2.9 cP to about 3.2 cP, about 3.0 cP to about 3.2 cP, about 2.8 cP to about 3.1 cP, about 2.9 cP to about 3.1 cP, about 3.0 cP to about 3.1 cP, about 2.8 cP to about 3.0 cP, or about 2.9 cP to about 3.0 cP.

In some embodiments, inhibitor particle formulations are administered at a T inhibitor KI dose in the range of about 10 mg to about 2500 mg and as a suspension having a viscosity in the range of about 2.7 cP to about 3.5 cP.

In some embodiments, the particle formulations are administered at a dose of the inhibitor in the range of about 10 to about 20 mg, or about 10 to about 50 mg, or about 25 to about 50 mg, or about 50 to about 100 mg, or about 75 to about 150 mg, or about 100 to about 250 mg, or about 200 to about 400 mg, or about 250 to about 500 mg, or about 300 to about 600 mg, or about 500 to about 1000 mg, or about 750 to about 1500 mg, or about 1000 to about 2000 mg, or about 1500 to about 2500 mg.

In certain aspects, an inhibitor of IGF-1R signaling may be formulated in biodegradable scaffolds such as, nanorods or microrods. In certain aspects, the microrods may optionally be formulated with a carrier. In certain aspects, the carrier is a matrix. In certain aspects, the matrix is selected from the group consisting of: collagen, gelatin, gluten, elastin, albumin, chitin, hyaluronic acid, cellulose, dextran, pectin, heparin, agarose, fibrin, alginate, carboxymethylcellulose, Matrigel™, hydrogel and organogel. In various aspects, the microrods are on average, each about 1 micron to 1,000 microns in length. In other aspects, the microrods each have a cross-sectional area of about A microns times B microns where A=1 micron to 1000 microns and B=1 micron to 1000 microns. In still other aspects, the microrods have a range of stiffness from about 1 kPa to about 1 GPa. In certain embodiments, the microrods have a shape of a regular or irregular polyhedron. In other embodiments, the microrods have a three-dimensional shape selected from the group consisting of: rod, cube, cone, cylinder, sphere, spiral, deltoid, asteroid, rhombus, parallelogram, trapezoid, cuboid, pyramid, prism, tetrahedron, pentahedron, hexahedron, septahedron, octahedron, nonahedron and decahedron, and irregular cross-sections. In certain aspects, the microrods have a stiffness ranging from about 1 kPa to about 1 GPa, and wherein the microrods have a rod shape having dimensions of A×B×L, wherein A is 1-30 μm, B is 1-30 μm, and L is 50-120 μm. In certain aspects, A is 15 pm, B is 15 pm, and L is 100 μm.

In one embodiment, the microrods are synthesized from one or more polymers. In other embodiments, polymer is selected from the group consisting of polylactic acid (PLA), polyglycolic acid (PGA), poly(ε-caprolactone) (PCL), poly(ethylene glycol) diacrylate (PEG-DA), poly(ethylene glycol) dimethacrylate (PEGDMA) and SU-8.

In another embodiment, the microrods are synthesized from one or more copolymers. In yet another embodiment, the copolymer is selected from the group consisting of poly(lactide-co-glycolide) (PLGA) and poly(DL-lactide-co-ε-caprolactone) (DLPLCL).

### Administration of Inhibitors of IGF-1 Signaling

In certain aspects, compositions or formulations of the inhibitors disclosed herein may be administered locally to a joint of a subject, such as, to an intra-articular space or a periarticular space of an osteoarthritic joint or a joint susceptible to developing OA, e.g., developing PTOA. In certain aspects, the administration may be a single administration, e.g., administration of a sustained release formulation of the IGF-1R signaling inhibitor or multiple administration. In certain aspects, compositions or formulations of the inhibitors disclosed herein may be administered to a joint of a subject by one or more intraarticular injections.

Any therapeutic amount of the inhibitor may be administered to a subject. In some cases, a formulation can be injected or infused into the human intra-articular space of the knee. Suitable volume can be easily adjusted by one of ordinary skill in this art for injections or delivery into other joints, such as the hip, shoulders, ankles, elbows, wrists, toes, fingers, and spinal facet joints.

It will be appreciated by those skilled in this art that administering may be performed by injection by syringe and/or other conventional modalities for delivering the compositions to a treatment site. The other conventional delivery modalities include catheters, infusion pumps, pen devices and the like.

In certain aspects, a scaffold or a matrix associated with the inhibitor of IGF-1R signaling may be injected or surgically placed into a joint of a subject in need thereof. In certain aspects, the inhibitor of IGF-1R signaling may be injected into a joint capsule of the subject.

The present disclosure describes methods comprising administration to a target site in a subject in need of treatment or prevention of OA, an effective amount of a pharmaceutical composition comprising one or more IGF-1 signaling inhibitors, wherein the one or more inhibitors are administered by one or more controlled release nanoparticle or microparticle systems. The administration may be localized and in certain cases formulated for sustained release over a period of time, e.g., at least 1 day, 3 days, 1 week, 2 weeks, or 3 weeks.

### Combination Treatment

The treatment or prevention of OA with one or more inhibitors of IGF-1 signaling may further include additional therapeutic molecules, such as, anti-analgesics, Anti-inflammatory agents, bone joint lubricating agents, and the like. These additional therapeutic molecules may be included in a pharmaceutical composition comprising an inhibitor of IGF-1R signaling for administration into the intraarticular space of a joint or may be administered in combination with the treatments disclosed herein. For example, simultaneously or sequentially.

Additional therapies that may be administered simultaneously or subsequently with the one or more inhibitors of IGF-1 signaling disclosed herein may be acetaminophen, non-steroidal anti-inflammatory drugs (NSAIDs) such as, naproxen, COX-2 selective inhibitors (such as celecoxib), or opioids.

In certain aspects, the subject may also be administered joint injections of glucocorticoids (such as hydrocortisone), hyaluronic acid, platelet-rich plasma (PRP) or placental tissue matrix (PTM).

### Subjects

According to certain embodiments, any subject having OA or susceptible to developing OA, e.g., PTOA may be treated by the disclosed methods. In certain aspects, the subject may be a human In certain aspects, the subject may be a mammal, such as, an equine, a bovine, a feline, or a canine.

### EXAMPLES

As can be appreciated from the disclosure provided above, the present disclosure has a wide variety of applications. Accordingly, the following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Those of skill in the art will readily recognize a variety of noncritical parameters that could be changed or modified to yield essentially similar results. Thus, the following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, dimensions, etc.) but some experimental errors and deviations should be accounted for.

### Example 1: Targeting IGF-1 Signaling Pathway for Prevention of Post-Traumatic Osteoarthritis (PTOA)

Introduction

Pathologic endochondral bone formation (EndoBF) can be triggered by injury resulting in post traumatic osteoarthritis (PTOA). Insulin-like growth factor-1 (IGF-1) signaling plays a fundamental role in regulating EndoBF during growth, but this can be deleterious in the adult joint. Our previous studies demonstrated that deletion of IGF-1R in chondrocytes prevents PTOA development. To investigate whether pharmacologic inhibition of this pathway could prevent and/or treat PTOA, we incorporated an IGF-1R inhibitor into polyethylene glycol dimethacrylate (PEG-DMA) hydrogel carriers (microrods) for prolonged local delivery in the joint space following ACL rupture and evaluated the effects of the inhibitor on PTOA development.

Abstract

Our previous studies showed that ablation of the insulin-like growth factor-1 receptor (IGF1R) in chondrocytes prevented the development of PTOA suggesting a potential means for its prevention. Here, we investigated the therapeutic role of the IGF1R inhibitor (NVP-AEW541) in PTOA development. NVP-AEW541 (IGF1Ri) was incorporated into the polyethylene glycol dimethacrylate (PEG-DMA) hydrogel carriers (microrods) for prolonged local delivery in the joint space following ACL rupture (ACLR). ACLR was achieved with a single bout of tibial compression overload (TCO) at 12 N of the right knee in 12 wks old C57BL6 mice. The left knee served as the control (CON). 1 day after the TCO, vehicle (Veh) or microrods loaded with inhibitor (IGF1Ri/rod) was injected into the injured knee. The CON and ACLR knees were analyzed 2 wks later Immunohistochemistry (IHC) demonstrated that ACLR induced phosphorylation of Erk in the Veh-treated injured knee, but the effect was blocked in the IGF1Ri-treated injured knee, verifying inhibition of IGF1 signaling. As determined by μCT, compared with the CON knee, ACLR decreased BV/TV (24%) in the subchondral bone, while the IGF1Ri/rod treatment (Tx) prevented the bone loss. Safranin O/Fast Green staining revealed erosion of the articular cartilage and formation of osteophytes in the Veh-treated injured knee, but these effects were not observed in the CON or IGF1Ri-treated injured knee. As indicated by IHC, ACLR increased the number of PCNA positive articular chondrocytes and MMP-13 production in the Veh-treated injured knee, but these effects were blunted in the IGF1Ri-treated injured knee. Moreover, ACLR increased the production of VEGF, resulting in vessel formation within articular cartilage (as indicated by CD31) in the Veh -treated injured knee, but these effects were blocked in the IGF1Ri-treated injured knee. To test the effects of IGF1Ri/microrods in chondrocytes in vitro, ATDC5 cells were treated by DMSO or IGF1Ri/microrod (4,000 microrods/0.5 ml), then further treated with PBS or IGF1. qPCR showed that delivery of the inhibitor blocked IGF1 stimulated expression of MMP-13 (65%) in chondrocytes. Our data indicate that IGF1 signaling plays deleterious roles in the development of PTOA by promoting abnormal articular chondrocyte proliferation and vascular invasion, while increasing matrix metallopeptidase production to damage articular cartilage and subchondral bone.

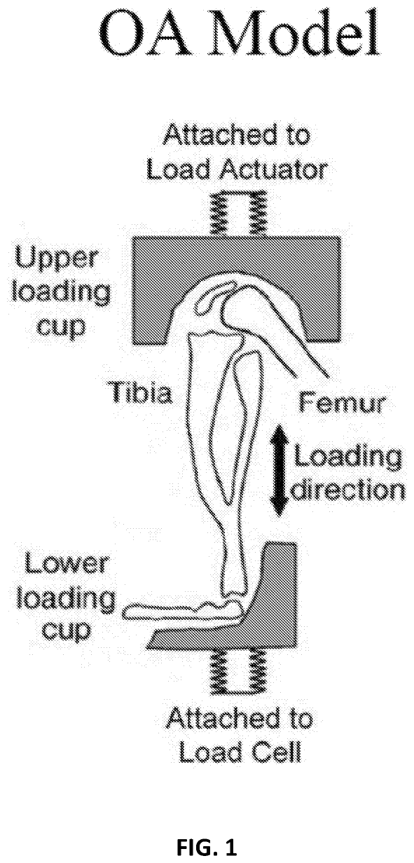

FIG. 1. Schematic of loading device for ACL rupture. The lower leg is placed between the upper and lower loading cups which are attached to the load actuator and load cell, respectively. A single 18N compression is applied. The resistance to the load is monitored by the attached computer, and the change in resistance documents the rupture of the ACL. This load did not result in fractures.

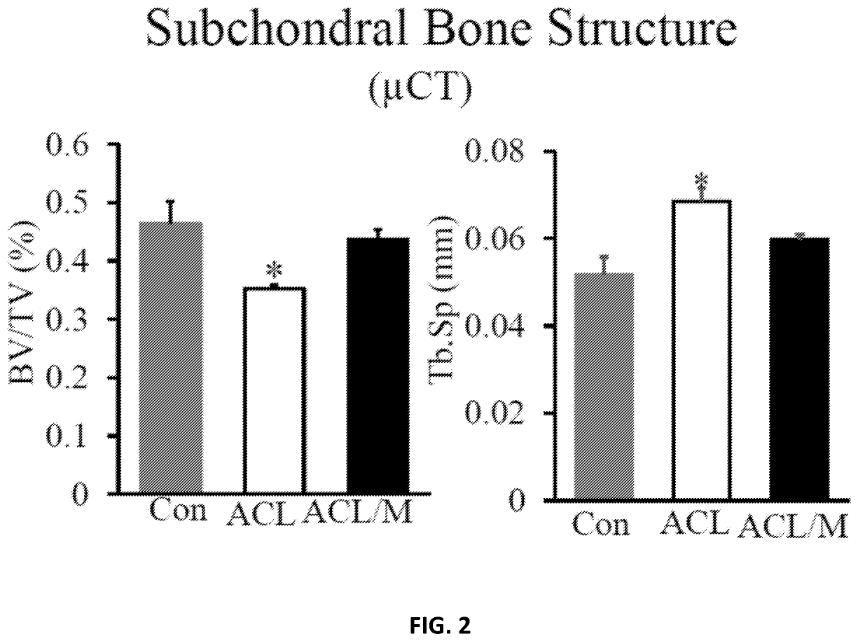

FIG. 2. IGF-IR inhibitor in microrods prevents subchondral bone loss during PTOA. ACL ruptures (injured) were performed in the right knee of 3M old mice. Vehicle or IGF-IR inhibitor in microrods (50 μl loaded with 2 mM NVP-AEW541) was injected into the injured knee one day after the injury. The left knee of each mouse served as normal control. Samples were collected 2 wks after injury. As determined by μCT, compared with the non injured knee (Con, gray bars), ACL rupture (ACL, open bars) decreased the trabecular bone volume (BV/TV, 24%), while increasing trabecular space (Tb. Sp, 36%). IGF-IR inhibitor in microrods (ACL/M, solid bars) blunted these effects. Results are expressed as mean ±SD. *p<0.05 vs. non injured knee.

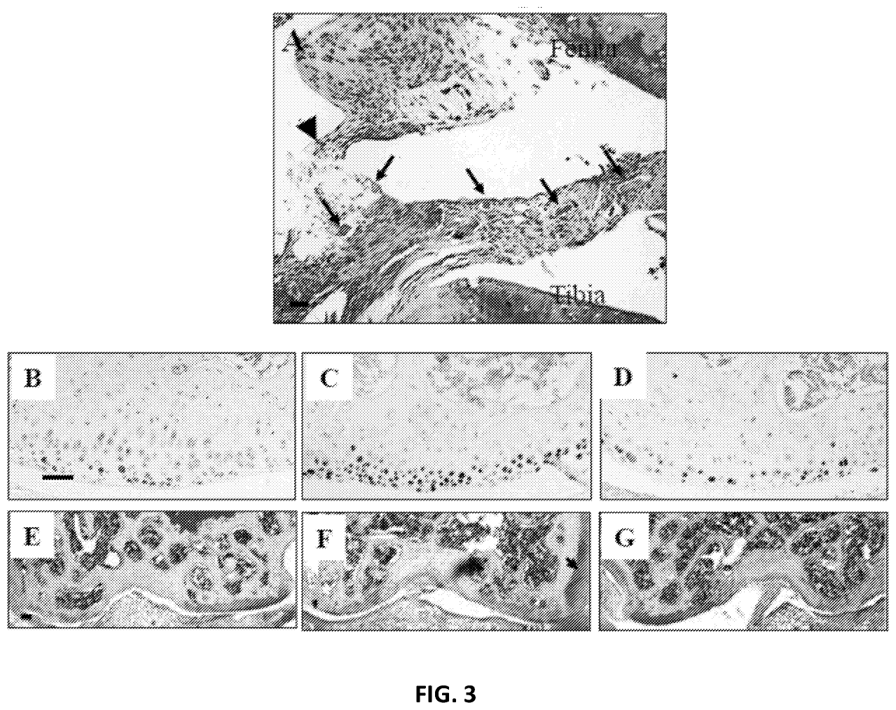

FIG. 3. IGF-1R inhibitor in microrods blocks activation of IGF-1R pathways & OA development induced by ACL rupture. A: In safranin O/Fast green (S/F) stained sections, microrods (arrows) are seen distributed in the knee capsule. Arrow head indicates the ruptured ACL. B-D Immunohistochemistry using phosphorylated ERK (pERK) antibody. Compared with the normal knee (B), pERK expression (brown) was increased in the articular chondrocytes of the injured knee (C), but these effects were blunted in the IGF-IR inhibitor treated knee (D). E-F: SF staining showed an osteophyte in injured knee (F, arrow), but not in the normal (E) and IGF-IR inhibitor treated knee (G). 10× in A-D, bars=50 μm; 2.5× in E-G. Bar=100 μm.

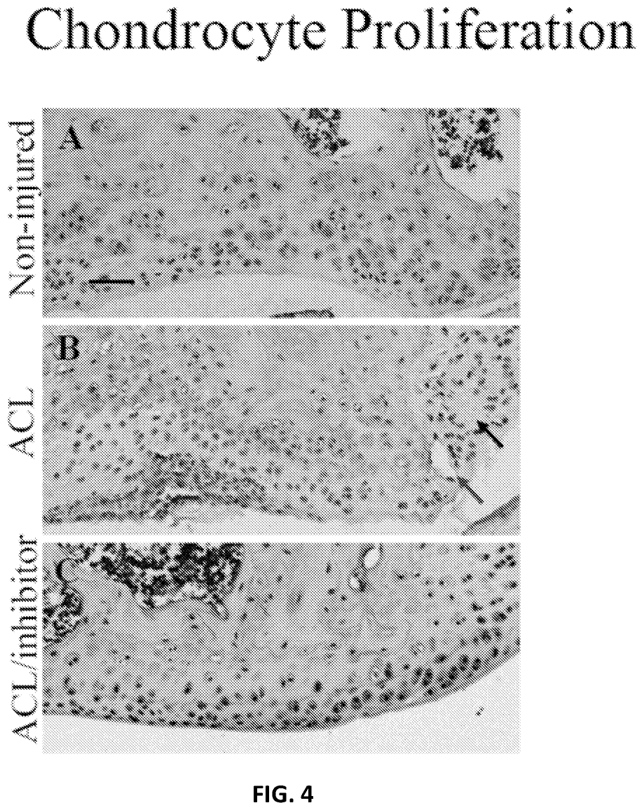

FIG. 4. IGF-1R inhibitor in microrods blunts ACL rupture induced articular chondrocyte proliferation. Immunohistochemistry using a PCNA antibody showed that injury (ACL rupture) increased the number of PCNA positive cells(brown) in the injured knee (B) compared with the non-injured knee (A). These effects were blunted by the IGF-1R inhibitor (C). In B: black arrow indicates an osteophyte, red arrow indicates the abnormal vasculature in cartilage. 10× in A-C bar=50 μm.

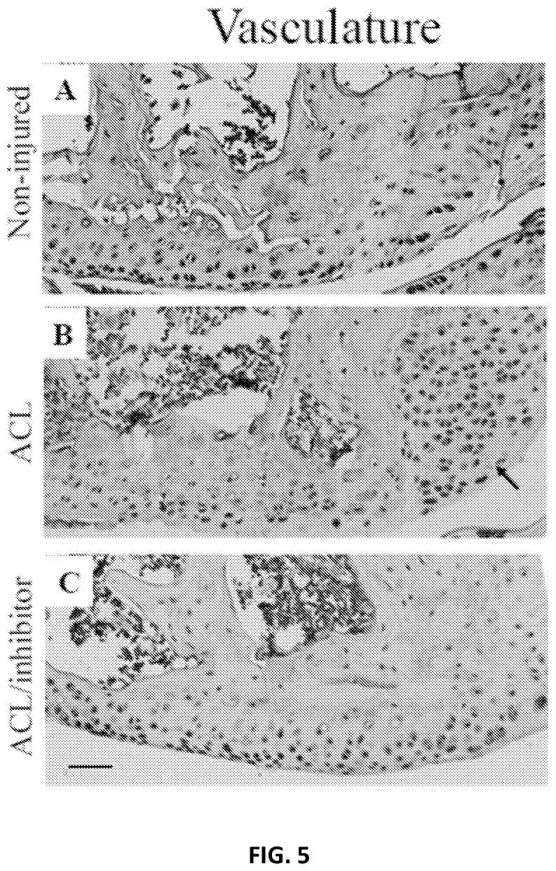

FIG. 5. IGF-1R in microrods decreases VEGF production induced by ACL rupture. Immunohistochemistry using an VEGF antibody (A-C) showed that injury (B) induced VEGF production (brown) when compared with the non injured knee (A). TheIGF-1R inhibitor blunted these effects. Arrow in B indicates an osteophyte. 10× in A-C, bar=50 μm.

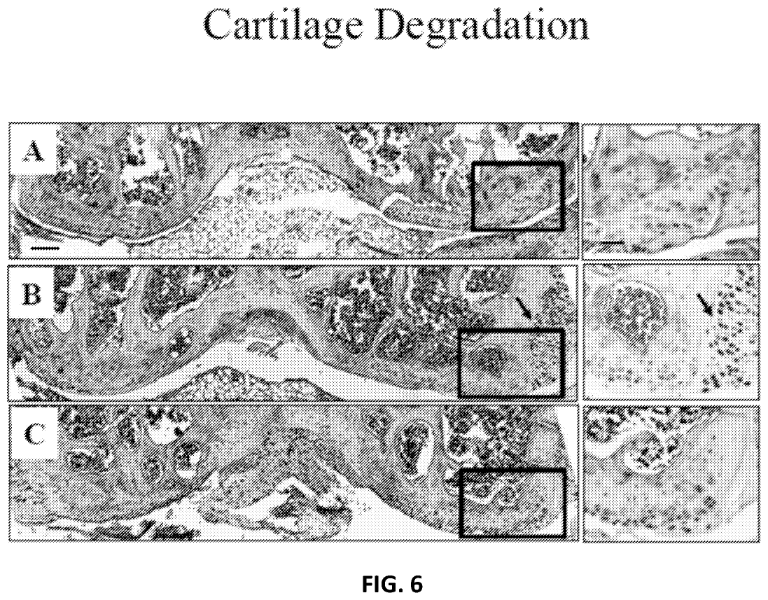

FIG. 6. IGF-1R inhibitor blocks MMP-13 production induced by ACL rupture. Immunohistochemistry using an MMP-13 antibody showed that compared with the non-injured knee (A), injury (B) induced MMP-13 production (brown) (A vs. B). The IGF-1R inhibitor blunts the induction. Pictures to the right are high magnification (10×, bar=50 μm) of the framed areas of the pictures on the left (5×, bar=100 μm).

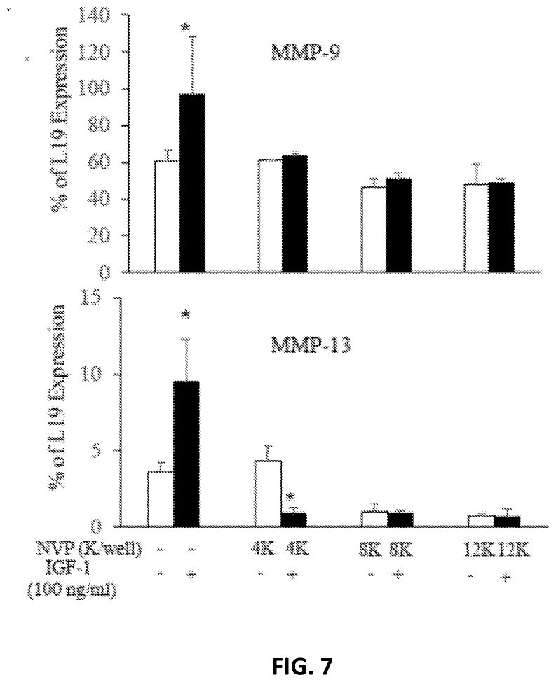

FIG. 7. IGF1R inhibitor incorporated into microrods blocks IGF1 induced MMP production. ATDC5 cells were treated with DMSO or IGF1R inhibitor (NVP) in microrods, then further treated with vehicle or IGF1 for 24 hrs. The mRNA levels of MMP-9 (upper panel) and MMP-13 (lower panel) were determined by quantitative real-time PCR. The dose of NVP (thousands of microrods/ well/0.5 ml) and IGF1 are shown on the x axis. Results are expressed as percentage of L-19 (house keeper gene) expression (mean±SD) of triplicate determinations. *: p<0.05 IGF-1 treated cultures vs. vehicle treated cultures.

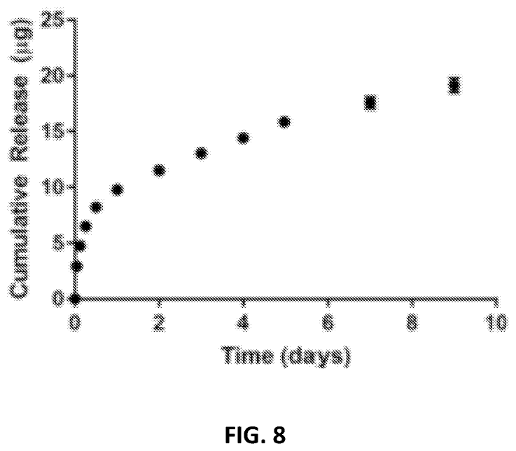

An extended release formulation of the specific IGF1R inhibitor NVP-AEW541 was used. In preliminary dose finding experiments this inhibitor was highly effective in blocking IGF1 stimulated MMP9 and MMP13 expression, inhibition that was retained when incorporated into the polyethylene glycol dimethacrylate (PEGDMA) hydrogel carrier (microrods) (data not shown). The elution profile of the inhibitor from the microrods is shown in FIG. 8. As expected, the elution is more rapid over the first 24 hrs, but continues for at least 9 days.

FIG. 8. Cumulative release of NVP-AEW541 from PEGDMA microrods. PEGDMA microrods were lyophilized in 1% Tween 20 and rehydrated in 2 mM (0.88 mg/mL) of NVP-AEW541 over 3 days. The microrods were washed 3× and resuspended in 150 μL of 0.1% Tween 20 in PBS. Over 9 days, samples were taken and the release buffer was replenished. Concentration of NVP-AEW541 in each sample was determined by absorbance at 285 nm. All release data shown were done in triplicate and presented as average±1 standard deviation. Each replicate contained 8×10⁵ microrods.

### SUMMARY

PTOA activates IGF-1 signaling. Inhibiting IGF-1R signaling by IGF-1R inhibitor incorporated in microrods decreased abnormal chondrocyte proliferation, inhibited abnormal vasculature and cartilage degradation and protected subchondral bone structure and prevented articular damage and osteophyte formation.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims. It is also to be understood that the terminology used herein is for the purpose of describing particular embodiments only, and is not intended to be limiting, since the scope of the present invention will be limited only by the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

What is claimed is:

1. A method for treating or preventing osteoarthritis in a subject in need thereof, the method comprising:
administering an inhibitor of insulin growth factor-1 receptor (IGF-1R) signaling into a joint of the subject in an amount effective to treat or prevent osteoarthritis.

2. The method of claim 1, wherein the method comprises treating osteoarthritis is a subject having osteoarthritis.

3. The method of claim 1, wherein the method comprises preventing osteoarthritis in a subject at risk for developing osteoarthritis.

4. The method of any of claims 1-3, wherein the subject has or is at risk for developing osteoarthritis in the knee.

5. The method of any of claims 1-4, wherein the osteoarthritis comprises post traumatic osteoarthritis (PTOA).

6. The method of any of claims 1-5, wherein the inhibitor of IGF-1R signaling inhibits activation of IGF-1R.

7. The method of claim 6, wherein the inhibitor inhibits binding of a ligand of IGF-1R to IGF-1R.

8. The method of claim 6, wherein the inhibitor inhibits phosphorylation of the IGF-1R.

9. The method of any of claims 1-5, wherein the inhibitor is an antibody that binds to IGF-1R.

10. The method of any of claims 1-6, wherein the inhibitor is a small molecule tyrosine kinase inhibitor.

11. The method of any of claims 1-6, wherein the inhibitor is NVP-AEW541.

12. The method of any of claims 1-11, wherein the administering comprises injecting the inhibitor into the joint of the subject.

13. The method of any of claims 1-11, wherein the administering comprises surgically placing the inhibitor into the joint of the subject.

14. The method of any of claims 1-13, wherein the inhibitor is associated with a scaffold.

15. The method of claim 14, wherein the scaffold comprises microrods associated with the inhibitor.
